(19) United States
(12) Patent Application Publication
(10) Pub. No.: US 2023/0063800 A1
(43) Pub. Date: Mar. 2, 2023

(54) DRAWING REVENUE SHARING SERVER, DRAWING REVENUE SHARING METHOD, AND COMPUTER PROGRAM

(71) Applicant: RFCAMP LTD., Gyonggi-do (KR)

(72) Inventors: Jae Hyung RYU, Gyeonggi-do (KR); Kwon Soo KIM, Seoul (KR); Ji Youn KIM, Gyeonggi-do (KR); Shang Gil LEE, Seoul (KR)

(73) Assignee: RFCAMP LTD., Gyonggi-do (KR)

(21) Appl. No.: 17/879,753

(22) Filed: Aug. 2, 2022

(30) Foreign Application Priority Data
Aug. 27, 2021 (KR) 10-2021-0113980

(51) Int. Cl. G06Q 40/00; B43L 13/00; G06F 3/04883

(52) U.S. Cl. CPC G06Q 40/12; B43L 13/00; G06F 3/04883

(57) ABSTRACT

A drawing revenue sharing method includes: acquiring response information and revenue information on drawing data generated by a first creator and a second creator; determining the revenue information on drawing data based on the response information on drawing data; and determining a revenue share value of the first creator and a revenue share value of the second creator by distributing the revenue information in a predetermined distribution ratio relationship.

### CROSS-REFERENCE TO RELATED APPLICATION

This application is based on and claims priority under 35 U.S.C. § 119 to Korean Patent Application No. 10-2021-0113980, filed on Aug. 27, 2021, in the Korean Intellectual Property Office, the disclosure of which is incorporated by reference herein in its entirety.

### BACKGROUND

### 1. Field

One or more embodiments relate to a drawing revenue sharing server, a drawing revenue sharing method, and a computer program. More specifically, one or more embodiments relate to a drawing revenue sharing server, a drawing revenue sharing method, and a computer program, which are capable of determining a revenue share value of each creator by considering the degree of contribution of each creator, the degree of influence on the surroundings, and the like with respect to drawing data created by a plurality of creators.

### 2. Description of the Related Art

The technical, temporal, and spatial limitations of creating a joint work by the collaboration of a plurality of creators on a digital canvas have been removed. “Joint work” refers to a work created jointly by two or more persons and the contribution of each person may not be separately used. Joint digital painting by a plurality of creators is expected to grow explosively in line with the rapid expansion of the digital art transaction market based on Non Fungible Token (NFT) in particular.

However, the reality is that revenue sharing standards among joint copyright holders are ambiguous and fall short of the development of technology.—When there is no special agreement between joint copyright holders, profit resulting from the use of a joint work is distributed to joint copyright holders according to the degree of contribution to the creation of the joint work. In this case, when the degree of contribution of joint copyright holders is unclear, it shall be presumed to be equal among the joint copyright holders. Therefore, along with the growth of digital art, it is necessary to establish a concrete and practical standard for distributing profit according to a contribution ratio in co-created digital paintings.

The above-mentioned background art is technical information possessed by the inventor for derivation of the present invention or acquired in the process of derivation of the present invention, and cannot necessarily be said to be a known technique disclosed to the general public prior to the filing of the present invention.

### SUMMARY

One or more embodiments include a drawing revenue sharing server, a drawing revenue sharing method, and a computer program, which are capable of distributing revenue from digital content created by a plurality of creators as revenue share values determined based on the degree of contribution to the digital content, the degree of influence on the surroundings, and the like.

In addition, one or more embodiments include a drawing revenue sharing server, a drawing revenue sharing method, and a computer program, which are capable of numerically calculating the degree of contribution and influence by each creator by analyzing drawing data for digital content and of determining a revenue share value of each creator based on the calculated degree of contribution and influence.

Additional aspects will be set forth in part in the description which follows and, in part, will be apparent from the description, or may be learned by practice of the presented embodiments of the disclosure.

According to one or more embodiments, a drawing revenue sharing method includes: acquiring response information and revenue information on drawing data generated by a first creator and a second creator; determining the revenue information on drawing data based on the response information on drawing data; and determining a revenue share value of the first creator and a revenue share value of the second creator by distributing the revenue information in a predetermined distribution ratio relationship.

The determining of the revenue share value includes determining a first distribution ratio relationship based on a degree of contribution by the first creator and the second creator with respect to the drawing data and distributing the revenue information in the first distribution ratio relationship.

A degree of contribution by the first creator may be determined based on touch events of the first creator during generation of the drawing data.

The touch events may include at least one of a working time, a number of strokes of a work brush, a movement distance of a work brush, a space occupancy level, pen pressure information, brush thickness information, touch brightness information, touch saturation information, and touch primary color information.

The determining of the revenue share value may include determining a second distribution ratio relationship based on a reproduction input for a drawing process in the response information on drawing data and distributing the revenue information in the second distribution ratio relationship.

A revenue share ratio of the first or second creator may be determined based on response information input with respect to the drawing data, and the second distribution ratio relationship may be determined such that revenue corresponding to the response information is distributed as revenue of the first or second creator related to an input time point of a response time point.

The determining of the revenue share value may include determining a third distribution ratio relationship based on a degree of influence on surroundings by the first creator or the second creator and distributing the revenue information in the third distribution ratio relationship.

The degree of influence on surroundings by the first creator or the second creator may be calculated based on psychological tendency information of the first creator or psychological tendency information of the second creator.

According to one or more embodiments, a drawing revenue sharing server includes: a processor, a communication unit, and a memory, wherein the processor is configured to execute stored computer readable instructions to acquire response information and revenue information on drawing data generated by a first creator and a second creator, determine the revenue information on drawing data based on the response information on drawing data, and determine a revenue share value of the first creator and a revenue share value of the second creator by distributing the revenue information in a predetermined distribution ratio relationship.

According to one or more embodiments, a computer program may be stored in a computer-readable storage medium for executing the method by using a computer.

In addition, another method and system for implementing the inventive concept of the present disclosure, and a computer-readable recording medium for recording a computer program for executing the method may be further provided.

Other aspects, features, and advantages other than those described above will be apparent from the following drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram of a network environment of a drawing revenue sharing system according to embodiments of the present disclosure;

FIG. 2 is a block diagram of a drawing revenue sharing server according to embodiments of the present disclosure, and FIG. 3 is a block diagram of a drawing revenue sharing unit according to embodiments of the present disclosure;

FIG. 4 is a flowchart of a drawing revenue sharing method according to embodiments of the present disclosure;

FIG. 5 is an example diagram of a table showing psychological type values of Creators 1 to 5;

FIG. 6 is an example diagram of a table showing type values of psychological type values for each type of continuous drawing; and

FIG. 7 is an example diagram of a table indicating parameters for determining the type of continuous drawing.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described more fully with reference to the accompanying drawings, in which exemplary embodiments of the present disclosure are shown.

As the present disclosure allows for various changes and numerous embodiments, particular embodiments will be illustrated in the drawings and described in detail in the written description. The attached drawings for illustrating exemplary embodiments are referred to in order to gain a sufficient understanding of the effects and features thereof, and methods for accomplishing the effects and features thereof. However, this is not intended to limit the present disclosure to particular modes of practice, and it is to be appreciated that all changes, equivalents, and substitutes that do not depart from the spirit and technical scope are encompassed in the present disclosure.

Hereinafter, the present disclosure will be described in detail by explaining exemplary embodiments of the present disclosure with reference to the attached drawings. Like reference numerals in the drawings denote like components, and thus their description will be omitted.

It will be understood that the terms used herein, such as “training,” “learning,” etc. are not intended to refer to mental actions such as human educational activities but refer to performing machine learning through computing according to procedures.

It will be understood that although the terms “first”, “second”, etc. may be used herein to describe various components, these components should not be limited by these terms. These components are only used to distinguish one component from another.

As used herein, the singular forms “a”, “an”, and “the” are intended to include the plural forms as well, unless the context clearly indicates otherwise.

It will be further understood that the terms “includes”, “has”, “including”, and/or “having” used herein specify the presence of stated features or components, but do not preclude the presence or addition of one or more other features or components.

Sizes of components in the drawings may be exaggerated for convenience of explanation. In other words, since sizes and thicknesses of components in the drawings are arbitrarily illustrated for convenience of explanation, the following embodiments are not limited thereto.

When a certain embodiment may be implemented differently, a specific process order may be performed differently from the described order. For example, two consecutively described processes may be performed substantially at the same time or performed in an order opposite to the described order.

Herein, revenue information is generated based on digital content, and may be generated by actions, such as trading, sharing, and permission of use of digital content. Transactions of digital content, such as sale, sharing, and permission to use, may be possible by various servers. Revenue information may be generated through a transaction action for digital content.

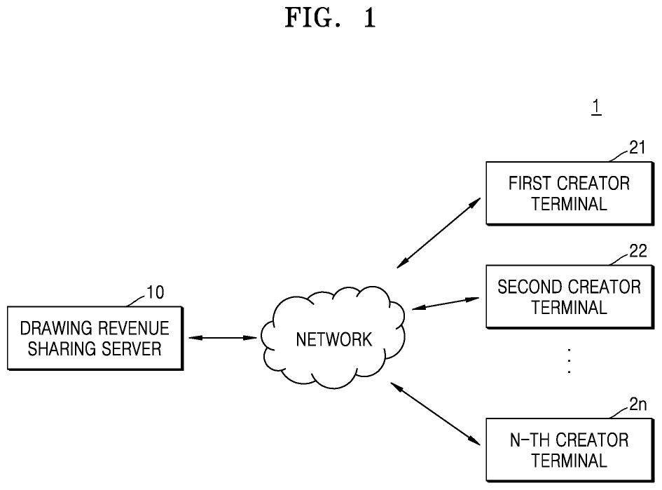

FIG. 1 is a diagram of a network environment of a drawing revenue sharing system 1 according to embodiments of the present disclosure.

The drawing revenue sharing system 1 may include a drawing revenue sharing server 10, a first creator terminal 21, a second creator terminal 22, . . . , and an n-th creator terminal 2n. The drawing revenue sharing server 10, the first creator terminal 21, the second creator terminal 22, . . . , and the n-th creator terminal 2n may be connected to one another through a network to transmit/receive data therebetween.

When revenue is generated through actions, such as disclosure, distribution, sale, and permission of use of digital content created by a plurality of creators, online, the drawing revenue sharing system 1 may distribute the generated revenue to a plurality of creators (i.e., the plurality of users) of the digital content. The drawing revenue sharing system 1 may determine revenue information by considering the degree of contribution by each creator, the degree of ripple by each creator, and the like only for digital content created by a plurality of creators. The drawing revenue sharing system 1 may distribute revenue information such as a revenue amount for digital content as a revenue share value of each creator. Drawing data may include digital content and data while the digital content is being generated. Here, the data may include data obtained during a drawing process. The degree of contribution by each creator may refer to a value for the degree to which the creative part of each creator contributed to the corresponding digital content. The degree of contribution by each creator may be determined based on the time created by each creator, the degree to which a portion created by each creator occupies, and the like. The degree of contribution by each creator may be determined by a predetermined rule. Rules determining the degree of contribution may be changeable by an administrator.

The drawing revenue sharing server 10 may determine revenue information and revenue share values for a plurality of creators of digital content, according to a received request. The drawing revenue sharing server 10 may acquire digital content, drawing data for digital content, and information about each creator from accessing another server or accessing a block chain system. The information about each creator may include psychological tendency information that is cumulatively managed for each creator. Here, the psychological tendency information may be determined from words input by each creator, drawings created by each creator, program usage behavior and attitude, and the like. The psychological tendency information may be current information or past information. The psychological tendency information may be information about a predetermined time point.

Users may access the drawing revenue sharing server 10 through the first creator terminal 21, the second creator terminal 22, . . . , and the n-th creator terminal 2n, which are of the creators.

At least one of the first creator terminal 21, the second creator terminal 22, . . . , and the n-th creator terminal 2n may generate digital content and transmit the digital content to the drawing revenue sharing server 10. At least one of the first creator terminal 21, the second creator terminal 22, . . . , and the n-th creator terminal 2n may acquire digital content, drawing data for when the digital content is generated, and data about the psychological tendency information of each creator, and transmit them to the drawing revenue sharing server 10. The digital content may be created by a plurality of creators. For example, final digital content may be generated by downloading, by the second creator terminal 22, digital content generated by the first creator terminal 21 and performing additional generating (completion). Digital content may be created by two or more creators. In this case, a first creator and a second creator may be creators of final digital content. At least one of the first creator terminal 21, the second creator terminal 22, . . . , and the n-th creator terminal 2n may acquire a time-series touch value at which digital content is generated while the digital content is generated, and may analyze the time-series touch value. At least one of the first creator terminal 21, the second creator terminal 22, . . . , and the n-th creator terminal 2n may acquire and store a working time by each creator, the number of strokes of a work brush by each creator, a movement distance of a work brush by each creator, a space occupancy level of digital content created by each creator, a pen pressure value of digital content created by each creator, characteristic information of a brush thickness value of digital content created by each creator, characteristic information of a color value of digital content created by each creator, and the like. Data stored in this way may be transmitted to the drawing revenue sharing server 10.

At least one of the first creator terminal 21, the second creator terminal 22, . . . , and the n-th creator terminal 2n may process registered digital content to share the registered digital content by adding the registered digital content to a feed or a page. At least one of the first creator terminal 21, the second creator terminal 22, . . . , and the n-th creator terminal 2n may process digital content to be shared by transmitting the digital content to a social network server associated with each creator's account.

At least one of the first creator terminal 21, the second creator terminal 22, . . . , and the n-th creator terminal 2n may input a sharing input, a like input, a dislike input, a purchase input, a selling input, a use permission input, etc. for digital content. Here, the sharing input, the liking input, the dislike input, the purchase input, the selling input, the use permission input, etc. may be input on an online where digital content is shared, or may be input through a link to the digital content.

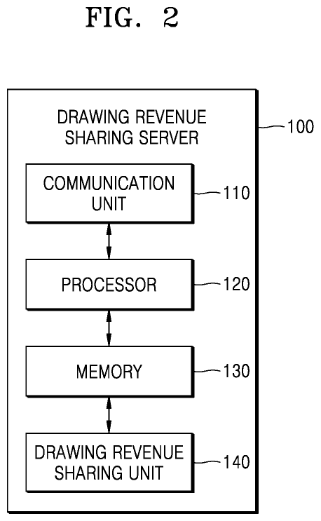

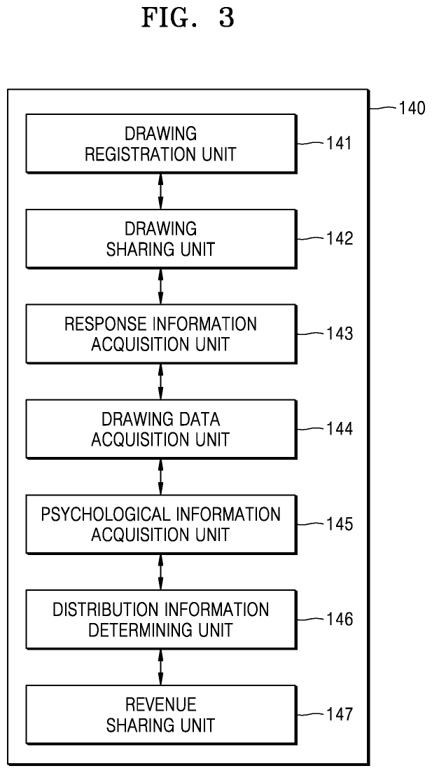

FIG. 2 is a block diagram of a drawing revenue sharing server 100 according to embodiments of the present disclosure, and FIG. 3 is a block diagram of a drawing revenue sharing unit 140 according to embodiments of the present disclosure.

The drawing revenue sharing server 100 refers to a computing device including a communication unit 110, a processor 120, a memory 130, and the drawing revenue sharing unit 140. Although the drawing revenue sharing server 100 is implemented as one device in FIG. 2, the drawing revenue sharing server 100 may be implemented as one or more computing devices, such as a distributed server and a distributed system. The processor 120 may read the drawing revenue sharing unit 140 and perform data processing. The communication unit 110 may transmit/receive necessary data under the control by the drawing revenue sharing unit 140. The communication unit 110 may provide a function for communicating with an external device through a network.

The memory 130 may be random access memory (RAM), read only memory (ROM), or a permanent mass storage device, such as a disk drive.

The drawing revenue sharing unit 140 may calculate revenue through an action of sharing drawing data generated by a plurality of creators and distribute the revenue according to the degree of contribution by each creator. The drawing data refers to data about digital content generated through an electronic device. The drawing data may include digital content and data about a process in which the digital content is generated. The drawing data may include digital content, a time-series digital content creation image, a time (working time) required for digital content creation, the number of strokes of a work brush, a movement distance of a work brush, a space occupancy level, a pen pressure value, a thickness value, and the brightness, saturation, and primary color degree of color values. The drawing data may be classified for each creator. For example, revenue may be distributed in the ratio of a time required for each creator to the total time required to draw corresponding content. The revenue may be distributed in the ratio of the number of strokes of each creator to the total number of strokes included in the content. The revenue may be distributed in the ratio of a stroke distance of each creator to the total stroke distance included in the content. The revenue may be distributed in the ratio of a space occupancy level occupied by each creator to the total space occupied by the content. The revenue may be distributed in the ratio of an average pen pressure of each creator to the average pen pressure of touches while drawing the content. The revenue may be distributed in the ratio of an average brightness value (saturation value, primary color value, etc.) of each creator to the average brightness value (saturation value, primary color value, etc.) of touches of the content. The drawing data may include digital content created by each creator, time-series generated images of digital content created by each creator, a working time by each creator, the number of strokes of a work brush by each creator, a movement distance of a work brush by each creator, a space occupancy level of digital content created by each creator, a pen pressure value of digital content created by each creator, characteristic information of a brush thickness value of digital content created by each creator, characteristic information of a color value of digital content created by each creator, and the like. The color value of digital content may include a brightness value, a saturation value, and a primary color ratio, and the like for a touch stroke constituting the digital content.

The drawing revenue sharing unit 140 may receive revenue information related to digital content, creator information, and the like. The drawing revenue sharing unit 140 may distribute revenue for digital content. In this case, the drawing revenue sharing unit 140 may distribute the revenue for digital content to each creator by considering various criteria for drawing data for the digital content, the degree of contribution of creators of the digital content, the degree of influence on the surroundings by the creators of the digital content, etc. For example, revenue may be distributed in the ratio of the number of feedbacks on parts drawn by each creator to the total number of feedbacks on content. In this case, the feedbacks may include positive feedback or comment feedback. Revenue may be distributed in the ratio of the number of reproduction inputs for parts drawn by each creator to the total number of reproduction inputs for drawing data.

A drawing registration unit 141 acquires and registers drawing data from an external device. The drawing registration unit 141 may register the drawing data to another external server. The external server to which the drawing data is registered may be a network-connected device, and may be implemented as a distributed server or a cloud server. The drawing registration unit 141 may store identification information of drawing data registered in a server and acquire the drawing data by using the identification information.

A drawing sharing unit 142 may process drawing data to be shared by one or more creator terminals. The drawing sharing unit 142 may generate interface data in which one or more pieces of drawing data are shared based on the drawing data registered in the server and a sharing interface of the drawing data. The sharing interface may include a shared information input area, a like information input area, a comment input area, and the like for the drawing data. In this case, the sharing interface may be for a function of sharing content to accounts existing online. The sharing interface may be provided by one or more external servers. The drawing sharing unit 142 may share drawing data and interface data including a sharing interface, and may count revenue information obtained as a result of sharing.

The revenue information may be created through drawing data and actions on networking where a sharing interface is provided and utilized. For example, revenue information may be created through advertisement information provided together with drawing data, or revenue information may be created through advertisement information additionally provided for a reproduction action on networking with respect to drawing data. Revenue information may be created while transferring rights to digital content, such as ownership and usage rights. The price for digital content may be determined according to a preset principle. In this case, the reproduction action may include an action (input) of reproducing a moving image about a process in which drawing data is drawn. The reproduction action may be input for a specific section during the drawing process. In addition, the revenue information may be created by trading, using, and the like of digital content. The revenue information may be acquired through one or more external servers.

When an input to the sharing interface provided for drawing data is acquired, a response information acquiring unit 143 may acquire data for the input to the sharing interface. Response information may include information on a shared input, a like input, a dislike input, a content confirmation input, etc. for drawing data, and may include the number of shared inputs, the number of like information inputs, the number of dislike information inputs, the number of reproductions of content, the length of reproduction time of content, and the like. In particular, response information on creators may be calculated based on a section reproduced by another creator in the content. For example, revenue may be distributed to a first creator in the ratio of the number of times a section drawn by the first creator is reproduced. The ratio of the number of times reproduced refers to a ratio of the number of times the corresponding section is reproduced to the total number of times reproduced. Revenue may be distributed to a second creator in the ratio of like information input for a section drawn by the second creator.

A drawing data acquisition unit 144 may acquire drawing data by using identification information of the drawing data. The drawing data may include factors for a touch stroke that generates digital content. The drawing data may include a coordinate value, a pen pressure value, a distance value, a speed value, a diameter value of a point, an inclination value of a touch means, an inclination value of a trace, a color value, a relative distance value, a relative speed value, a time value, a relative pen pressure value, a relative stop time, a relative thickness (dot diameter size) of a stroke, and the like, which are the factors for a touch stroke. The drawing data acquisition unit 144 may acquire drawing data from other creator terminals or an external server device. In this case, the drawing data may include digital content, time-series generated images of digital content created by each creator, a working time by each creator, the number of strokes of a work brush by each creator, a movement distance of a work brush by each creator, a space occupancy level of digital content created by each creator, a pen pressure value of digital content created by each creator, characteristic information of a brush thickness value of digital content created by each creator, characteristic information of a color value of digital content created by each creator, and the like. The drawing data acquisition unit 144 may acquire digital content and time-series touch values, from which the digital content is generated, from other creator terminals, and generate drawing data based on the digital content and the time-series touch values. As the time-series touch values, there may be a pen pressure value, a speed value, and a color value. After the digital content is completed, the drawing data acquisition unit 144 may acquire data about the digital content and generate drawing data for distribution of revenue information. The drawing data may be generated while the digital content is generated using a program installed in a creator terminal. The drawing data generated for the digital content may be acquired together with the digital content.

In the present embodiment, the space occupancy level may be calculated based on occupancy levels for an occupied pixel occupied by a touch and a non-occupied pixel in digital content. The occupancy level of the occupied pixel may be calculated from factors of the occupied pixel. The occupancy level may be determined as a sum of the occupancy levels of each pixel that is proportional to at least one of a pen pressure value, a color value, and an occupied time value of each pixel. In this case, the occupancy level may be calculated by considering an occupied area of the occupancy level and the number of occupied pixels. The occupancy level may further consider the occupancy level of the occupied pixel as well as the occupancy level of the non-occupied pixel. The occupancy level of the non-occupied pixel may be calculated based on the pen pressure value, color value, distance value, and occupancy time value of an adjacent occupancy pixel. Additionally, the occupancy level of the non-occupied pixel may further consider the occupancy level of an adjacent occupancy pixel. The occupancy level of the drawing data may be calculated by considering the occupancy level of the occupied pixel and the occupancy level of the non-occupied pixel. In this case, the space occupancy level for each creator may be calculated by calculating an occupancy level for a part created by each creator.

A psychological information acquisition unit 145 may calculate psychological tendency information on drawing data for digital content and acquire psychological tendency information of each creator, based on drawing data for a part (created part) drawn by each creator in the digital content. Additionally, the psychological information acquisition unit 145 may acquire the psychological tendency information of each creator by using user behavior data, setting data, and the like for a certain application. The relational expression between the user behavior data and the psychological tendency information or the relational expression between the setting data and the psychological tendency information may be stored in advance.

The psychological information acquisition unit 145 may determine the psychological tendency information of each creator based on each attribute included in drawing data for a part drawn by each creator and a relational expression between factors included in the drawing data. The relational expression or change pattern for the drawing data may include a time-series distance change pattern, a speed change pattern, a pen pressure change pattern, a thickness change pattern, a stop change pattern, or the like. Optionally, the relational expression or change pattern for the drawing data may include a speed change pattern, a pen pressure change pattern, a thickness change pattern, a stop time change pattern, or the like according to a distance change. The relational expression or change pattern may include a relational expression between a speed and a pen pressure value, a relational expression between a movement distance and a pen pressure value, and the like. The relational expression or change pattern for the drawing data may be determined for each creator, and information on the psychological tendency of each creator according to the relational expression or change pattern of each creator may be determined. In another embodiment, the psychological tendency information of the first creator and the psychological tendency information of the second creator may be relatively determined based on a relational expression between the attribute of the drawing data of the first creator and the attribute of the drawing data of the second creator.

Additionally, the psychological information acquisition unit 145 may determine the psychological tendency information of each creator by considering emotional content selected by each creator and the degree of movement and the speed of movement while the emotional content is selected. The psychological tendency information of each creator may be determined by one or more emotional contents selected by each creator and a path, movement degree, movement speed, etc. of a user input for selecting one or more emotional contents. The emotional content may be content designated as a value of a stimulus level and a value of a positive level, or may be preset by an administrator. The emotional content may be set as a word, but is not limited thereto. The psychological tendency information of each creator may be determined based on the degree of movement and the movement speed of each creator while a predetermined number of emotional contents are selected from among a plurality of provided emotional contents. A high degree of movement or a high movement speed while selecting emotional content may mean that extroversion, energy, etc. are high among the psychological tendency information of the creator.

Additionally, the psychological information acquisition unit 145 may determine the psychological tendency information of each creator based on the behavior data of each creator for an application. The factors included in the behavior data are for a given application, and may include an access location, the proportion of access, the frequency of access, whether to approve one-line touch, whether to approve login, whether to disclose a selected emotional word, information on the time until selecting a first word related to a selection input of an emotional word set, information on the ratio of repeatedly selected emotional words, dominant emotions set by the emotional words, emotional index, display coordinate values of the selected emotional words, and the like. Based on factors of the behavior data and a combination of the factors, the psychological tendency information of each creator may be determined. Information on the psychological tendency information corresponding to the factors of the behavior data may be stored in advance.

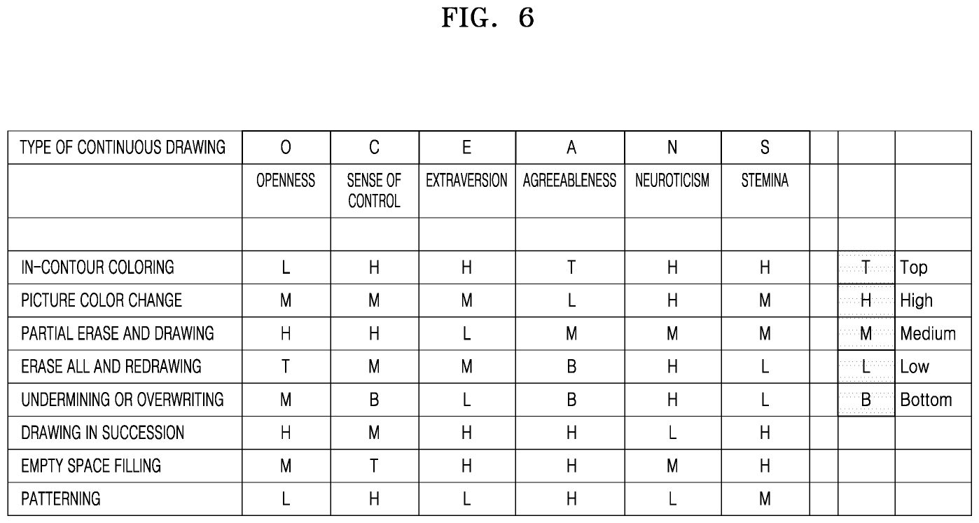

In another embodiment, the psychological information acquisition unit 145 may determine the psychological tendency information of each creator according to the generation type of first drawing data. When the first drawing data is data generated by the second creator continuing to draw with respect to data generated by a first creator, the psychological tendency information of the first creator and the second creator may be determined based on the type of continuous drawing. A table between the type of continuous drawing input and values for each type of a psychological type value may be defined as shown in FIG. 6.

A distribution information determining unit 146 may distribute revenue information, created by sharing the first drawing data created by the first creator and the second creator, according to drawing data of a part created by each creator. The distribution information determining unit 146 may determine a revenue share value of the first creator, based on a working time of a part created by the first creator, the number of strokes of a work brush, a movement distance of a work brush, a space occupancy level of digital content, a pen pressure value of digital content, characteristic information of a brush thickness value of digital content, characteristic information of a color value of digital content, and the like in the first drawing data.

Each creator's revenue share value (ratio) may be determined based on each creator's working time/total working time, each creator's (touch) strokes/total strokes, each creator's stroke distance/total stroke distance, each creator's space occupancy level/total space, (average pen pressure—average pen pressure of each creator)/average pen pressure, (total average brush thickness-average brush thickness of each creator)/total average thickness, (total average brightness value-average brightness value of each creator)/total average brightness value, (total average saturation value-average saturation value of each creator)/total average saturation value, (total average primary color value-average primary color value of each creator)/total average primary color value, the number of like inputs or the number of comment inputs in a specific section during a picture reproduction process/total number of like inputs or total number of comment inputs, the number of reproductions in a specific section during a picture reproduction process/total number of reproductions in the entire section, and openness, sense of control, extroversion, agreeableness, neuroticism, stamina, and the like in the psychological tendency information of each creator. However, the present disclosure is not limited thereto, and each creator's revenue share value (ratio) may be determined based on a combination of various factors. More specifically, the revenue of a creator who contributed more to the dominant psychological tendency information of content may be set higher. The dominant psychological tendency information may refer to a disposition determined as a high value among psychological tendency information determined through the completed drawing data. For example, when openness in the psychological tendency information of content is the highest, the revenue of a creator having the higher openness in the psychological tendency information of a part created by each creator may be set higher. When sense of control in the psychological tendency information of content is the highest, the revenue of a creator having the higher sense of control in the psychological tendency information of a part created by each creator may be set higher. The psychological tendency information of content may include information on openness, sense of control, extroversion, agreeableness, neuroticism, stamina, and the like, and may be expressed in the order of openness, sense of control, extroversion, agreeableness, neuroticism, and stamina.

The distribution information determining unit 146 may determine a revenue share value of the second creator, based on a working time, the number of strokes of a work brush, a movement distance of a work brush, a space occupancy level of digital content, a pen pressure value of digital content, characteristic information of a brush thickness value of digital content, characteristic information of a color value of digital content, and the like for a part created by the second creator. The distribution information determining unit 146 may determine a revenue share value of each creator as a ratio of the working time by the first creator or the working time by the second creator to the total working time of the first drawing data. The distribution information determining unit 146 may determine a revenue share value for each attribute by applying the above process to the working time, the number of strokes of the work brush, the movement distance of a work brush, the space occupancy level, the pen pressure value, the brush thickness value, the brightness value, the saturation value, the primary color value, and the like, and may determine a revenue share value for each creator by combining revenue share values for attributes. In this case, different weight values may be applied to the attributes, or the same weight value may be applied to the attributes. A weight value applied to each attribute may be recalculated through a past revenue share value, a revenue share history, and the like.

Accordingly, the distribution information determining unit 146 may calculate the degree of contribution of each creator through at least one of a working time of a part created by each creator, the number of strokes of a brush, a movement distance of a brush, a space occupancy level, a pen pressure value, a brush thickness, a brightness value, a saturation value, and a primary color value, and may determine a revenue share value corresponding to the degree of contribution of each creator. At least one of the working time, the number of strokes of a brush, the movement distance of a brush, the space occupancy level, the pen pressure value, the brush thickness, the brightness value, the saturation value, and the primary color value may be acquired through creative actions of each creator while digital content is created. The accuracy and types of acquired information may be changed by considering capabilities (CPU performance, memory performance, network performance, etc.) of a device during when the creative actions are performed. The distribution information determining unit 146 may determine a contribution degree or a revenue share value by considering attributes obtainable from drawing data.

The distribution information determining unit 146 may distribute revenue information based on the degree of influence on the surroundings by each creator on the first drawing data. The distribution information determining unit 146 may calculate the degree of influence on the surroundings by each creator based on the psychological tendency information of a part generated by each creator or the psychological tendency information of each creator. The distribution information determining unit 146 may calculate the degree of influence on the surroundings by each creator based on the psychological tendency information of each creator determined by the psychological information acquisition unit 145.

In this case, the psychological tendency information may be determined by dividing the psychological tendency information into preset parameters, such as openness, sense of control, extraversion, agreeableness, neuroticism, and stamina. The degree of influence on the surroundings may be determined by applying a predetermined weight value to each parameter in the psychological tendency information. For example, the degree of influence on the surroundings may be determined to be a value proportional to openness and extraversion and may be determined to be a value inversely proportional to neuroticism, but is not limited thereto and may be determined by various relational expressions. The degree of influence on the surroundings may be set as a relational expression for parameters included in the psychological tendency information.

The distribution information determining unit 146 may determine a revenue share value for a creator by considering the psychological tendency of a part created by the creator. The distribution information determining unit 146 may determine a revenue share value for a creator by considering the psychological tendency of a part created by the creator in the psychological tendency information of content. When openness in the psychological tendency information of content is the highest, the revenue share value of a creator whose psychological tendency of a created part has openness may be set high. The higher the openness of the creator in the psychological tendency information, the higher the revenue share value for the creator may be, but the present disclosure is not limited thereto. The lower the sense of control of the creator, the higher the revenue share value for the creator may be, but the present disclosure is not limited thereto. The higher the extroversion of the creator, the higher the revenue share value for the creator may be, but the present disclosure is not limited thereto. The lower the agreeableness of the creator, the higher the revenue share value for the creator may be, but the present disclosure is not limited thereto. The higher the neuroticism of the creator, the higher the revenue share value for the creator may be, but the present disclosure is not limited thereto. The lower the stamina of the creator, the higher the revenue share value for the creator may be, but the present disclosure is not limited thereto. The aforementioned cases are only examples, and various relationships may be established. In this way, the revenue share value of each creator may be determined through a pattern between parameters included in the psychological tendency information of each creator.

A revenue sharing unit 147 may determine a revenue share value for distributing revenue information on drawing data to each creator, and may distribute the revenue information according to the revenue share value. In this case, the drawing data may be digital content itself, and may include digital content and information related to the digital content. The digital content may be a picture, a sound source, or the like, and may refer to visual data, auditory data, or audiovisual data. The digital content may have a digital form and be generated by an electronic device, but is not limited thereto and may be anything having a digital form.

The revenue sharing unit 147 may acquire information on creators for drawing data and determine revenue information as revenue share values for the creators. The revenue sharing unit 147 may distribute the revenue information to each creator's account according to the determined revenue share values.

The revenue sharing unit 147 may transmit and store the calculated revenue share values to an external server.

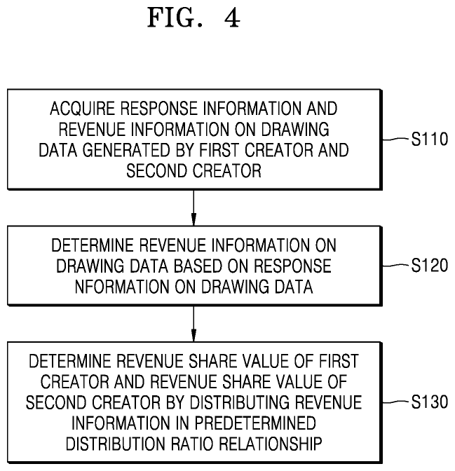

FIG. 4 is a flowchart of a drawing revenue sharing method according to embodiments of the present disclosure.

In operation S110, the drawing revenue sharing server 100 may acquire response information and revenue information on drawing data generated by a first creator and a second creator.

In operation S120, the drawing revenue sharing server 100 may determine revenue information on the drawing data based on response information on the drawing data.

In operation S130, the drawing revenue sharing server 100 may determine the revenue share value of the first creator and the revenue share value of the second creator by distributing the revenue information in a predetermined distribution ratio relationship.

The drawing revenue sharing server 100 may determine a first distribution ratio relationship based on the degree of contribution by the first creator and the second creator with respect to the drawing data, and may distribute the revenue information in the first distribution ratio relationship. The distribution ratio relationship refers to the distribution ratios to creators.

The degree of contribution by each creator may be determined based on touch events (strokes) by each creator during generation of the drawing data. In this case, the touch events may be determined by considering at least one of a working time, the number of strokes of a work brush (touch), a movement distance of the work touch, a space occupancy level, pen pressure information, brush thickness information, a brightness value of the touch, a saturation value of the touch, and a primary color value of the touch. The degree of contribution may be determined by the drawing information acquisition unit 144.

The drawing revenue sharing server 100 may determine a second distribution ratio relationship based on a reproduction input for a drawing process in response information on the drawing data, and may distribute the revenue information in the second distribution ratio relationship. The first or second distribution ratio relationship may include a relative distribution value between creators, a relationship between distribution values, and the like.

When a reproduction input for the drawing process is obtained, the drawing revenue sharing server 100 may process the reproduction input such that more revenue information according to the reproduction input is distributed to a creator of a section reproduced by the reproduction input. In this case, when the reproduction input is repeatedly made, a revenue share value may increase by the number of times the reproduction input is repeatedly made. The drawing revenue sharing server 100 may determine a revenue share ratio of the first or second creator based on response information input with respect to the drawing data, but may determine a second revenue distribution relationship such that revenue corresponding to the response information is distributed as the revenue of the first or second creator related to an input time point of a response time point.

The drawing revenue sharing server 100 may determine a third distribution ratio relationship based on the degree of influence on the surroundings by the first or second creator, and may distribute the revenue information in the third distribution ratio relationship. The degree of influence on the surroundings may be calculated based on the psychological tendency information of each creator (user). Herein, a user may refer to a creator.

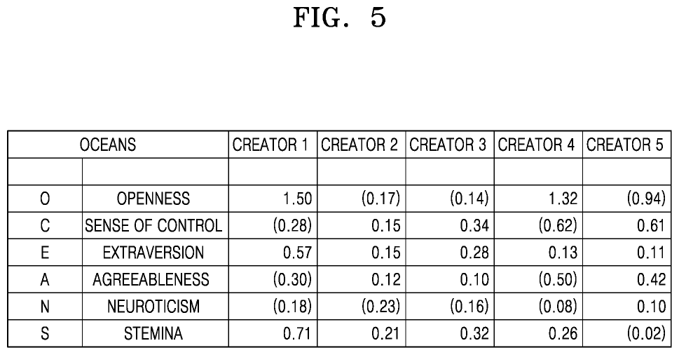

FIG. 5 is an example diagram of a table showing psychological type values of Creators 1 to 5;

The psychological type values of each creator may be defined as parameters (type values) of openness, sense of control, extraversion, agreeableness, neuroticism, and stamina, as shown in FIG. 5.

In the case that first drawing data is generated by Creators 1 and 2, when the openness of Creator 1 is compared to the openness of Creator 2, Creator 1 may have a higher revenue share value than Creator 2. When the sense of control of Creator 1 is compared to the sense of control of Creator 2, Creator 1 may have a higher revenue share value than Creator 2. When the extroversion of Creator 1 is compared to the extroversion of Creator 2, Creator 1 may have a higher revenue share value than Creator 2. When the agreeableness of Creator 1 is compared to the agreeableness of Creator 2, Creator 1 may have a higher revenue share value than User 2. When the neuroticism of Creator 1 is compared to the neuroticism of Creator 2, Creator 1 may have a higher revenue share value than Creator 2. When the stamina of Creator 1 is compared to the stamina of Creator 2, Creator 2 may have a higher revenue share value than Creator 1. When the psychological type values of Creator 1 are compared to the psychological type values of Creator 2, Creator 1 may have a higher revenue share value than Creator 2. A revenue share value of Creator 1 and a revenue share value of Creator 2 may be relatively determined with a result of adding up a difference in openness, a difference in sense of control, a difference in extraversion, a difference in agreeableness, a difference in neuroticism, a difference in stamina, and the like by considering a logic that determines revenue share values, and revenue for Creators 1 and 2 may be determined based on the revenue share values of Creators 1 and 2. Revenue for Creator 1 and revenue for Creator 2 may be respectively accumulated in Creator 1's account and Creator 2's account or separate accounts.

In the case that second drawing data is generated by Creators 3 and 4, when the openness of Creator 3 is compared to the openness of Creator 4, Creator 4 may have a higher revenue share value than Creator 3. When the sense of control of Creator 3 is compared to the sense of control of Creator 4, Creator 4 may have a higher revenue share value than Creator 3. When the extroversion of Creator 3 is compared to the extroversion of Creator 4, Creator 3 may have a higher revenue share value than Creator 4. When the agreeableness of Creator 3 is compared to the agreeableness of Creator 4, Creator 4 may have a higher revenue share value than Creator 3. When the neuroticism of Creator 3 is compared to the neuroticism of Creator 4, Creator 4 may have a higher revenue share value than Creator 3. When the stamina of Creator 3 is compared to the stamina of Creator 4, Creator 4 may have a higher revenue share value than Creator 3. When the psychological type values of Creator 3 are compared to the psychological type values of Creator 4, Creator 4 may have a higher revenue share value than Creator 3. A revenue share value of Creator 3 and a revenue share value of Creator 4 may be relatively determined with a result of adding up a difference in openness, a difference in sense of control, a difference in extraversion, a difference in agreeableness, a difference in neuroticism, and the like by considering a logic that determines revenue share values, and revenue for Creators 3 and 4 may be determined based on the revenue share values of Creators 3 and 4. Revenue for Creator 3 and revenue for Creator 4 may be respectively accumulated in Creator 3's account and Creator 4's account or separate accounts. In the above example, a case in which drawing data is generated by two creators is described, but drawing data may be generated by three or more creators. Even in this case, the revenue share values of the creators may be determined by considering the degree of contribution and the degree of ripple.

FIG. 6 is an example diagram of a table showing type values of psychological type values for each type of continuous drawing.

Among a plurality of creators of drawing data, the type values of the psychological type values for each type of continuous drawing of the drawing data may be expressed in five levels. Through the type of continuous drawing, the psychological type values of each creator may be determined. As shown in FIG. 6, the parameters (type values) of each psychological type value may be expressed in a plurality of levels. Specifically, the five levels are Top(T), High(H), Medium(M), Low(L), and Bottom(B). The levels of the type values may be converted into one of T, H, M, L, and B according to the size and degree thereof. T may denote a first range having a largest level size, H may denote a second range having a next largest level size, M may denote a third range having an intermediate level size, L may denote a fourth range having a level size smaller than the intermediate level size, and B may denote a fifth range having a smallest level size.

When the type of continuous drawing input is “in-contour coloring”, among the psychological type values, openness may be L, sense of control may be H, extraversion may be H, agreeableness may be T, and neuroticism may be H, and stamina may be H.

When the type of continuous drawing input is “picture color change”, openness may be M, sense of control may be M, extroversion may be M, agreeableness may be L, neuroticism may be H, and stamina may be M.

When the type of continuous drawing input is “partial erase and drawing”, openness may be H, sense of control may be H, extroversion may be L, agreeableness may be M, neuroticism may be M, and stamina may be M.

When the type of continuous drawing input is “erase all and redrawing”, openness may be T, sense of control may be M, extroversion may be M, agreeableness may be B, neuroticism may be H, and stamina may be L.

When the type of continuous drawing input is “undermining or overwriting”, openness may be M, sense of control may be B, extroversion may be L, agreeableness may be B, neuroticism may be H, and stamina may be L.

When the type of continuous drawing input is “continuous drawing”, openness may be H, sense of control may be M, extraversion may be H, agreeableness may be H, neuroticism may be L, and stamina may be H.

When the type of continuous drawing input is “empty space filling”, openness may be M, sense of control may be T, extraversion may be H, agreeableness may be H, neuroticism may be M, and stamina may be H.

When the type of continuous drawing input is “patterning”, openness may be L, sense of control may be H, extraversion may be L, agreeableness may be H, neuroticism may be L, and stamina may be M.

The type of continuous drawing input of the drawing data may not be one of the above. For example, when the type of continuous drawing input of the first drawing data is “empty space filling” and “continuous drawing”, a psychological type of a creator of the first drawing data may be determined based on a psychological type value according to the type of “empty space filling” and a psychological type value according to the type of “continuous drawing”. In this case, a psychological type of the party of the continuous drawing input may be determined by a psychological type value for the continuous drawing input.

When the first drawing data is generated by the first creator and the second creator and is generated as the second creator performs “continuous drawing” on drawing data drawn by the first creator, a psychological type value of the first creator may be determined based on drawing data generated by the first creator, and a psychological type value of the second creator may be determined based on the type of continuous drawing according to a part continuously drawn by the second creator.

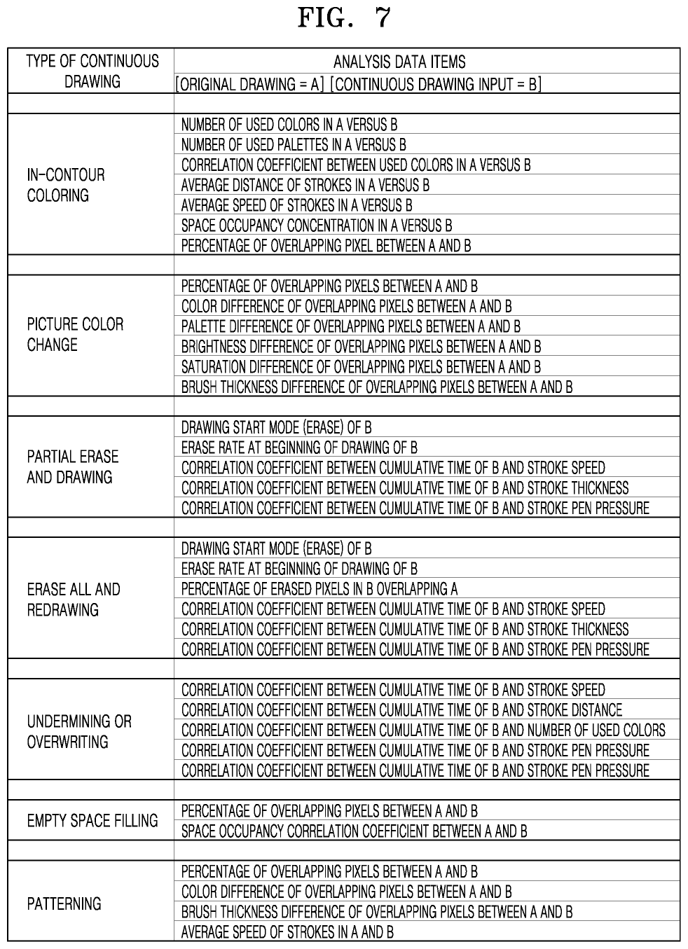

FIG. 7 is an example diagram of a table indicating parameters for determining the type of continuous drawing.

The device described above may be implemented as a hardware component, a software component, and/or a combination of the hardware component and the software component. For example, the device and components described in the embodiments may be implemented using one or more general purpose or special purpose computers, such as a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and one or more software applications running on the OS. The processing device may also access, store, manipulate, process, and generate data in response to execution of the software. For convenience of understanding, although one processing device is sometimes described as being used, one of ordinary skill in the art will recognize that the processing device may include a plurality of processing elements and/or a plurality of types of processing elements. For example, the processing device may include a plurality of processors or one processor and one controller. Other processing configurations, such as parallel processors, are also possible.

The software may include a computer program, code, instructions, or a combination of one or more of the foregoing, and may configure the processing device so that the processing device can operate as intended, or to independently or collectively give instructions to the processing device. The software and/or the data may be permanently or temporarily embodied in any type of machine, component, physical device, virtual equipment, computer storage media or devices, or transmitted signal waves, such that the software and/or the data is interpreted by the processing apparatus or provides an instruction or data to the processing device. The software may be distributed over a networked computer system and stored or executed in a distributed manner. The software and the data may be stored in one or more computer readable recording media.

The method according to the embodiment may be embodied in the form of program instructions that can be executed through various computer means and recorded in a computer-readable medium. The computer-readable medium may include program instructions, data files, data structures, and the like separately or in combinations. The program instructions to be recorded on the computer-readable recording medium may be specially designed and configured for the embodiments or may be well-known to and available to one of ordinary skill in the art of computer software. Examples of the computer-readable recording medium include magnetic media (e.g., hard disks, floppy disks, magnetic tapes, etc.), optical media (e.g., CD-ROMs, or DVDs), magneto-optical media (e.g., floptical disks), and hardware devices specifically configured to store and execute program instructions (e.g., ROM, RAM, flash memories, etc.) Examples of the program instructions are advanced language code that can be executed by a computer by using an interpreter or the like as well as machine language code made by a compiler. The hardware devices can be configured to function as one or more software modules so as to perform operations according to examples, or vice versa.

While the embodiments have been particularly shown and described with reference to examples thereof, it will be understood by those of ordinary skill in the art that various changes in form and details may be made therein without departing from the spirit and scope of the present disclosure as defined by the following claims. For example, an appropriate result may be attained even when the above-described techniques are performed in a different order from the above-described method, and/or components, such as the above-described system, structure, device, and circuit, are coupled or combined in a different form from the above-described methods or substituted for or replaced by other components or equivalents thereof.

Therefore, other implementations, other embodiments, and equivalents to the claims are also within the scope of the following claims.

According to one or more embodiments described above, a drawing revenue sharing server, a drawing revenue sharing method, and a computer program, which are capable of distributing revenue from digital content created by a plurality of creators as revenue share values determined based on the degree of contribution to the digital content, the degree of influence on the surroundings, and the like, may be provided.

In addition, according to one or more embodiments described above, a drawing revenue sharing server, a drawing revenue sharing method, and a computer program, which are capable of numerically calculating the degree of contribution and influence by each creator by analyzing drawing data for digital content and of determining a revenue share value of each creator based on the calculated degree of contribution and influence, may be provided.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A drawing revenue sharing method comprising:
acquiring, by a drawing revenue sharing server, response information and revenue information on drawing data generated by a first creator and a second creator;
determining, by the drawing revenue sharing server, the revenue information on drawing data based on the response information on drawing data; and
determining, by the drawing revenue sharing server, a revenue share value of the first creator and a revenue share value of the second creator by distributing the revenue information in a predetermined distribution ratio relationship.

2. The drawing revenue sharing method of claim 1, wherein the determining of the revenue share value comprises determining a first distribution ratio relationship based on a degree of contribution by the first creator and the second creator with respect to the drawing data and distributing the revenue information in the first distribution ratio relationship.

3. The drawing revenue sharing method of claim 2, wherein a degree of contribution by the first creator is determined based on touch events of the first creator during generation of the drawing data.

4. The drawing revenue sharing method of claim 3, wherein the touch events include at least one of a working time, a number of strokes of a work brush, a movement distance of a work brush, a space occupancy level, pen pressure information, brush thickness information, touch brightness information, touch saturation information, and touch primary color information.

5. The drawing revenue sharing method of claim 1, wherein the determining of the revenue share value comprises determining a second distribution ratio relationship based on a reproduction input for a drawing process in the response information on drawing data and distributing the revenue information in the second distribution ratio relationship.

6. The drawing revenue sharing method of claim 5, wherein a revenue share ratio of the first or second creator is determined based on response information input with respect to the drawing data, and the second distribution ratio relationship is determined such that revenue corresponding to the response information is distributed as revenue of the first or second creator related to an input time point of a response time point.

7. The drawing revenue sharing method of claim 1, wherein the determining of the revenue share value comprises determining a third distribution ratio relationship based on a degree of influence on surroundings by the first creator or the second creator and distributing the revenue information in the third distribution ratio relationship.

8. The drawing revenue sharing method of claim 7, wherein the degree of influence on surroundings by the first creator or the second creator is calculated based on psychological tendency information of the first creator or psychological tendency information of the second creator.

9. A drawing revenue sharing server comprising:
a processor, a communication unit, and a memory,
wherein the processor is configured to execute stored computer-readable instructions to acquire response information and revenue information on drawing data generated by a first creator and a second creator, determine the revenue information on drawing data based on the response information on drawing data, and determine a revenue share value of the first creator and a revenue share value of the second creator by distributing the revenue information in a predetermined distribution ratio relationship.

10. A non-transitory tangible computer-readable recording medium on which a program for performing the drawing revenue sharing method of claim 1 is recorded.
